(19) United States
(12) Patent Application Publication
(10) Pub. No.: US 2013/0096460 A1
(43) Pub. Date: Apr. 18, 2013

(54) SKIN GAS COLLECTION TOOL

(71) Applicant: CANON KABUSHIKI KAISHA, Kamakura-shi (JP)

(72) Inventor: Takashi Ikeda, Kamakura-shi (JP)

(73) Assignee: CANON KABUSHIKI KAISHA, Tokyo (JP)

(21) Appl. No.: 13/629,083

(22) Filed: Sep. 27, 2012

(30) Foreign Application Priority Data
Oct. 13, 2011 (JP) 2011-225448

(51) Int. Cl. A61B 5/00

(52) U.S. Cl. 600/573

(57) ABSTRACT

A skin gas collection tool includes a casing forming a tubular opening section, a tubular flexible film member arranged along the inner lateral surface of the casing and a gas collection vessel gastightly linked to the casing. The tubular flexible film member is held at the opposite ends thereof in gastight contact with the inner lateral surface of the casing to form a sealed space between itself and the inner lateral surface of the casing and forced to expand toward the center of the tube as a fluid substance is fed into the sealed space. The tubular flexible film member has a thickness and an elasticity satisfying a predetermined relation.

### BACKGROUND OF THE INVENTION

1. Field of the Invention

The present invention relates to a tool for collecting skin gas emanated from a skin surface.

2. Description of the Related Art

Researches and studies have been conducted on the correlation between the substances in the body of a living creature (animal) and the components of the gas emanated from the skin surface of the living creature (to be referred to as “skin gas” hereinafter) for the purpose of diagnosing the condition of the inside of the living creature such as health condition by observing the components and their concentrations of the gas emanated from the skin surface of the living creature. For example, there was a research conducted on the correlation between the acetone concentration in the gas components emanated from the skin surface of a living creature and the concentration of β-hydroxy lactic acid in the blood of the living creature (see Non-Patent Document 1: Kazutoshi Nose, Takaharu Kondo, Shuhki Araki and Takao Tsuda: The Japan Society for Analytical Chemistry: Vol. 54, p. 161 (2005).

Skin gas is collected from a living creature by means of a collection tool in some form in order to measure the concentration of each of all or some of the components of skin gas. Ordinarily, the body part for collection of skin gas is a hand, a foot, a finger, etc. The body part may simply be inserted into a vessel to collect skin gas. However, care should be taken so as not to allow air get into the vessel and mix with skin gas. For example, an apparatus for collecting gas emanated from the skin surface of a hand of a subject including a sampling bag having a tubular opening section that operates as insertion port has been proposed. The sampling bag is provided on the inner surface of the tubular opening section with an air bag made of a flexible material as gas emanating body part holding member for holding the inner surface of the tubular opening section of the sampling bag in gastight contact with the wrist of the subject when the gas emanating body part (hand) is inserted into the sampling bag from the tubular opening section and air is injected into the air bag at a pressure not impeding the flow of blood, that is, at a pressure higher than the atmospheric pressure (substantially one atmosphere) by 30 to 40 mmHg has been proposed (see Patent Document 1: Japanese Patent Application Laid-Open No. 2007-155385).

The holding member in the form of an air bag described in the above-cited patent document is highly convenient because the holding member can hold a base section (wrist or the like) of the gas emanating body part in position with ease. However, the holding member in the form of an air bag is covered at the outer periphery thereof by a rigid tubular opening section casing and, as air is injected into the holding member, the holding member is forced to expand toward the center of the tubular opening section. Then, as a result, the length of the surface of the holding member in the intra-tubular peripheral direction is reduced to give rise to one or more wrinkles (folds) in the longitudinal direction of the tube in the area where the surface of the holding member is held in gastight contact with the skin of the wrist and its neighboring parts and air can get into the sampling bag through the wrinkled parts of the holding member.

### SUMMARY OF THE INVENTION

In view of the above-identified problem, the present invention provides a skin gas collection tool including: a casing forming a tubular opening section for insertion of a body part to be used for skin gas collection (to be referred to as skin gas collection body part hereinafter); a tubular flexible film member arranged along the inner lateral surface of the casing; and a gas collection vessel gastightly linked to the casing; the tubular flexible film member being held at the opposite ends thereof in gastight contact with the inner lateral surface of the casing to form a sealed space between itself and the inner lateral surface of the casing; the casing having a fluid substance feed/discharge port for feeding a fluid substance into or discharging a fluid substance from the sealed space; the tubular flexible film member being forced to expand toward the center of the tube as a fluid substance is fed into the sealed space to thereby hold the tubular flexible film member in gastight contact with the skin gas collection body part inserted into the tubular opening section; the product σt, or the result of multiplication of the thickness t of the tubular flexible film member and the force σ required to stretch a cube of a unit length of the tubular flexible film member by a unit length in a direction satisfying the requirement of the inequality formula (1) shown below and expressed in terms of the limit pressure Pₘₐₓ of the fluid substance, the length s of the tubular flexible film member, the radius R of the tubular opening, the radius r of the region of the skin gas collection body part held in gastight contact with the tubular flexible film member as approximated by a circular cylinder and the number N of the folds produced on the flexible film member when the skin gas collection body part is held in position:

$$σ t \leq \frac{{P}_{max} s}{\sqrt{2} ( π - 2 ) {\{ \begin{matrix}{( R + r )}^{2} {sin}^{2} ( \frac{90 R - 90 r}{NR} ) + \\ {( R - r )}^{2} {cos}^{2} ( \frac{90 R - 90 r}{NR} )\end{matrix} \}}^{\frac{1}{2}}} . \qquad ( 1 )$$

Thus, skin gas can be collected from a subject, or a living creature, with ease by means of a skin gas collection tool according to the present invention without giving the living creature any physical or mental pain. Further features of the present invention will become apparent from the following description of exemplary embodiments with reference to the attached drawings.

### BRIEF DESCRIPTION OF THE DRAWINGS

FIG. 1 is a schematic illustration of a skin gas collection tool according to the present invention.

FIG. 2 is a schematic illustration of the tubular flexible film member of a skin gas collection tool according to the present invention showing a state where the member is expanded toward the center of the tube.

FIG. 3 is a schematic cross sectional view taken along plane T-T′ in FIG. 2.

FIG. 4 is a geometric expression of the flexibility requirement to be met by the tubular flexible film member of a skin gas collection tool according to the present invention.

FIG. 5 is a schematic illustration of a groove formed in the casing of a skin gas collection tool according to the present invention.

### DESCRIPTION OF THE EMBODIMENTS

Preferred embodiments of the present invention will now be described in detail in accordance with the accompanying drawings.

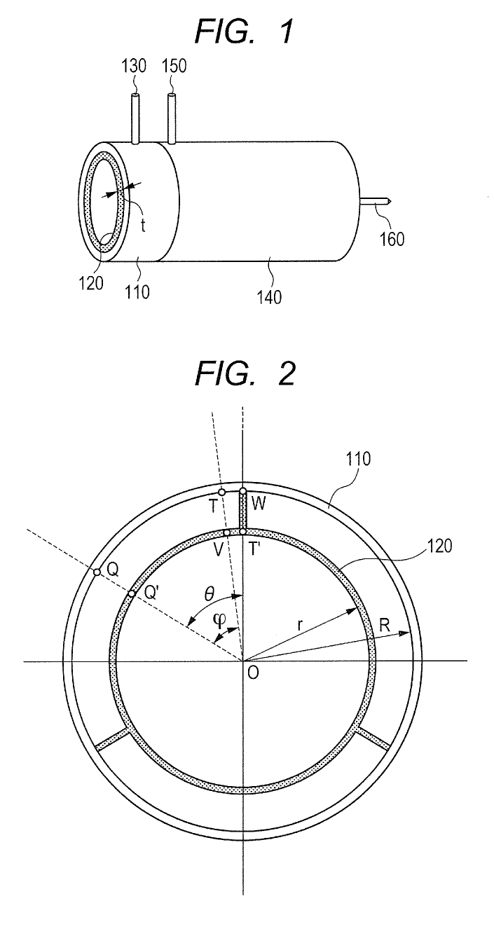

FIG. 1 is a schematic illustration of a skin gas collection tool according to the present invention. A skin gas collection tool according to the present invention includes a casing 110 forming a tubular opening section for insertion of a skin gas collection body part such as a hand, a feet or a finger, a tubular flexible film member 120 arranged along the inner lateral surface of the casing and a gas collection vessel 140 gastightly linked to the casing. The tubular flexible film member is held at the opposite ends thereof in gastight contact with the inner lateral surface of the casing to form a sealed space between itself and the inner lateral surface of the casing. On the other hand, the casing has a fluid substance feed/discharge port 130 so as to feed a fluid substance into or discharging a fluid substance from the sealed space with the pressure of the fluid substance controlled. The tubular flexible film member is forced to expand toward the center of the tube as a fluid substance is fed into the sealed space to thereby hold the tubular flexible film member in gastight contact with the skin gas collection body part inserted into the tubular opening section.

Thus, both the casing 110 and the fluid substance feed/discharge port 130 are required to have such a degree of strength that does not allow the casing 110 and the port 130 to be deformed remarkably when pressurized by a fluid substance. Additionally, both the casing 110 and the fluid substance feed/discharge port 130 need to be made of a material that is poorly gas-releasable/permeable and also poorly skin-gas adsorbable. While there are various such materials including glass, metals and plastic materials, Teflon (registered trademark) resin and stainless steel can particularly suitably be employed.

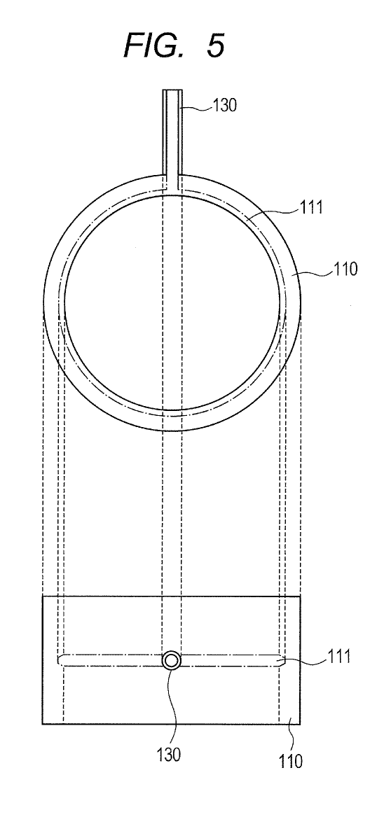

Meanwhile, if a skin gas collection body part presses the tubular flexible film member 120 against the fluid substance feed/discharge port 130 hard and closes the former, no fluid substance can flow into the sealed space so that the tubular flexible film member may not be expanded. Therefore, preferably, a plurality of fluid substance feed/discharge ports are provided or one or more grooves 111 are formed on the inner lateral surface of the casing 110 so as to cross the fluid substance feed/discharge port as shown in FIG. 5 and prevent the fluid substance feed/discharge port from being closed by the tubular flexible film member 120. While a single circular groove is formed in FIG. 5, the groove may not necessarily have such a profile. Additionally, the groove may have any length so long as the groove does not allow the fluid substance feed/discharge port to be closed with ease.

Preferably, the fluid substance is chemically stable relative to the tubular flexible film member and the fluid substance feed/discharge port 130 and shows a high degree of fluidity so that the pressure being applied to the skin gas collection body part can be made to fall to the level of atmospheric pressure instantaneously when abnormal pressure appears. The fluid substance may be air for instance.

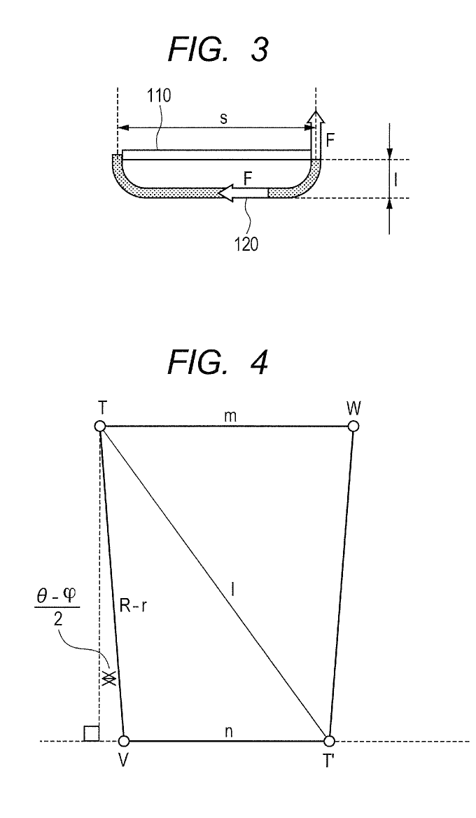

The tubular flexible film member 120 is required to show such a characteristic that the product σt, or the result of multiplication of the thickness t of the tubular flexible film member and the force σ required to stretch a cube of a unit length of the material of the tubular flexible film member by a unit length in a (film surface) direction satisfies the requirement of the inequality formula (1) shown below. In the formula (1), Pₘₐₓ is the limit pressure of the fluid substance that is the limit value of the pressure (the difference between the atmospheric pressure) applied to force the tubular flexible film member to expand or the pressure upper limit for not physically and mentally damaging the subject living creature and s is the length of the tubular flexible film member, while R is the radius of the tubular opening (or the value obtained by subtracting the thickness t of the film member from the radius of the tubular flexible film member to be more accurate), r is the radius of the region of the skin gas collection body part held in gastight contact with the tubular flexible film member as approximated by a circular cylinder, which is the smallest radius of the region in principle (see FIGS. 2 and 3) and N is the number of the folds (wrinkles) produced on the flexible film member. Note that the number of wrinkles depends on the material of the tubular flexible film member and the outer diameter of the region that is held in gastight contact as described above.

$$σ t \leq \frac{{P}_{max} s}{\sqrt{2} ( π - 2 ) {\{ \begin{matrix}{( R + r )}^{2} {sin}^{2} ( \frac{90 R - 90 r}{NR} ) + \\ {( R - r )}^{2} {cos}^{2} ( \frac{90 R - 90 r}{NR} )\end{matrix} \}}^{\frac{1}{2}}} . \qquad ( 1 )$$

Now, that the characteristic of the tubular flexible film member is limited by the mathematical formula (1) will be described below in detail.

FIG. 2 schematically illustrates a state where a skin gas collection tool according to the present invention holds a circular cylindrical object having a radius r. In other words, the region of the skin gas collection body part that is held by the tubular flexible film member 120 is approximated by a circular cylinder. More specifically, FIG. 2 schematically shows that the tubular flexible film member is forced to expand toward the center thereof and folds (wrinkles) are formed at three positions as a fluid substance is injected under pressure higher than the atmospheric pressure into the sealed space formed between the tubular flexible film member 120 and the casing 110. The number of wrinkles varies depending on the size of the region held by the tubular flexible film member and the characteristic of the tubular flexible film member. Therefore, the number of wrinkles is generally expressed by N in the following description. Assume that the pressure in the sealed space is equal to the atmospheric pressure and the intersection of the tubular flexible film member 120 in a not deformed state and the prolongation of line segment OQ′ is defined as Q. Note that point Q′ is the middle point of two neighboring folds. As the tubular flexible film member 120 is expanded to hold a predetermined region of the skin gas collection body part, the point Q moves to the position of the point Q′. On the other hand, T′ is the contact point of a fold and the skin gas collection body part. T′ is located at point T when the tubular flexible film member 120 is not expanded and the length of circular arc QT and that of circular arc Q′T′ may safely be assumed to be equal to each other with πr/N. The tubular flexible film member 120 needs to be deformed to the largest extent at point T (point T′) and hold the skin gas collection body part without any gap. In other words, the pressure to be applied to the tubular flexible film member in order to move point T to point T′ is the pressure required to hold the skin gas collection body part without any gap.

FIG. 3 is a schematic cross sectional view taken along plane T-T′ in FIG. 2. FIG. 3 illustrates that the tubular flexible film member 120 is subjected to tensile stress F on the cross section and forced to expand as a fluid substance is injected into it under pressure higher than the atmospheric pressure. Now, let us consider to what extent the tubular flexible film member needs to be expanded in order to hold the skin gas collection body part without any gap. FIG. 4 illustrates the size of the isosceles trapezoid TWT′V shown in FIG. 2. As seen from FIG. 4, the length l of the line segment TT′ is expressed by formula (2) shown below.

$$l = {\{ {( \frac{m + n}{2} )}^{2} + {( R - r )}^{2} {cos}^{2} ( \frac{θ - ϕ}{2} ) \}}^{\frac{1}{2}} . \qquad ( 2 )$$

The expansion Δs of the tubular flexible film member 120 is (πl −2l) and hence expressed by formula (3) shown below from formula (2).

$$\begin{matrix}Δ s = ( π - 2 ) {\{ {( \frac{m + n}{2} )}^{2} + {( R - r )}^{2} {cos}^{2} ( \frac{θ - ϕ}{2} ) \}}^{\frac{1}{2}} \\ = ( π - 2 ) {\{ {( R + r )}^{2} {sin}^{2} ( \frac{90 R - 90 r}{NR} ) + {( R - r )}^{2} {cos}^{2} ( \frac{90 R - 90 r}{NR} ) \}}^{\frac{1}{2}} .\end{matrix} \qquad ( 3 )$$

Force F required to expand the tubular flexible film member having a length s by Δs and working in parallel with the direction of expanding the member (tensile stress that is produced accordingly) is expressed by formula (4) shown below.

$$F = \frac{t σΔ s}{s} . \qquad ( 4 )$$

Forces F are applied to the tubular flexible film member in directions orthogonal to each other as shown in FIG. 3. The pressure P applied to the tubular flexible film member by the fluid substance injected into the member equals the resultant force of the two forces F and hence formula (5) shown below is led out.

$$\begin{matrix}P = \frac{\sqrt{2} t σΔ s}{s} \\ = \frac{\sqrt{2} ( π - 2 ) t σ {\{ \begin{matrix}{( R + r )}^{2} {sin}^{2} ( \frac{90 R - 90 r}{NR} ) + \\ {( R - r )}^{2} {cos}^{2} ( \frac{90 R - 90 r}{NR} )\end{matrix} \}}^{\frac{1}{2}}}{s} .\end{matrix} \qquad ( 5 )$$

Formula (6) is led out by solving formula (5) for σt.

$$σ t = \frac{Ps}{\sqrt{2} ( π - 2 ) {\{ \begin{matrix}{( R + r )}^{2} {sin}^{2} ( \frac{90 R - 90 r}{NR} ) + \\ {( R - r )}^{2} {cos}^{2} ( \frac{90 R - 90 r}{NR} )\end{matrix} \}}^{\frac{1}{2}}} . \qquad ( 6 )$$

When holding a skin gas collection body part of a living creature by applying pressure to the body part, application of strong pressure can cause a discomforting sensation in the creature. Therefore, there is an upper limit to applicable pressure. If the applicable pressure upper limit is expressed by Pₘₐₓ, σt needs to satisfy the requirement of the inequality formula (1).

A skin gas collection vessel 140 is gastightly fitted to the casing 110 in order to cover the skin gas collection body part and collect skin gas in a manner that does not allow air to get into the collected skin gas. The skin gas collection vessel is required to be made of a material that is poorly gas-releasable/permeable and also poorly skin-gas adsorbable. The volume capacity of the skin gas collection vessel may be either variable or invariable. A material such as vinyl fluoride film may be used for the skin gas collection vessel to make the volume capacity thereof variable. On the other hand, a material such as Teflon (registered trademark) resin or stainless steel may be used for the skin gas collection vessel to make volume capacity thereof invariable.

Skin gas collection methods can be roughly classified into flow systems by means of which skin gas is collected while carrier gas is being flown and sealed systems that involve no gas inflow/outflow. To realize a flow system, a skin gas collection vessel 140 is provided with a carrier gas injection port 150. The carrier gas injection port 150 is preferably arranged at a position located close to skin gas collection body part insertion port 110 to make the injected carrier gas into contact with the surface of the skin gas collection body part over a broad area. A gas output port 160 is provided to take out gas from the inside of the skin gas collection vessel 140. In a flow system, a gas collection tool may be fitted to the front end of the gas output port 160 in order to inject gas into a measurement/analysis instrument or the gas output port may be directly connected to a measurement/analysis instrument. A sealed system, on the other hand, is plugged or provided with a valve in order to prevent air from getting into the inside. As in the case of the fluid substance feed/discharge port 130, both the gas injection port 150 and the gas output port 160 are preferably made of Teflon (registered trademark) resin or stainless steel.

The limit pressure Pₘₐₓ that can be applied to a tubular flexible film member was looked into for human beings as subjects. Wrists and fingers of hands were selected as body parts for checking compression. As a result, it was found that the broadest tolerance range for compression is provided when the third finger (middle finger) is held at the basal joint thereof. The pressure that was applied to the basal joint of the third finger started causing a discomfort in subjects at about 0.0114 MPa in average and the standard deviation was about 0.00135 MPa. When the subjects show a normal distribution for pressures that causes a discomfort in them and the pressure at which 98% of the subjects would not feel any discomfort is selected as limit pressure Pₘₐₓ, the limit pressure is preferably 0.0087 MPa, which is the value obtained by subtracting twice of the standard deviation from the average value.

### Examples

### Example-1

A casing 110, a fluid substance feed/discharge port 130, a skin gas collection vessel 140, a carrier gas injection port 150 and a gas output port 160 were prepared with SUS316. The inner diameter 2R of the insertion port was made equal to 24 mm. A tube having a thickness t of 1.3 mm and an outer diameter of 24mm and made of styrene-ethylene-butadiene copolymer was used for a tubular flexible film member 120. The force σ required to expand a cube whose sides had a unit length and that was made of the material of the tubular flexible film member by a unit length was 0.013 MPa/mm. Thus, at was 0.0169 MPa.

The second finger whose basal joint had a diameter 2r of about 20 mm was inserted into a skin gas collection tool according to the present invention and compressed air of 0.008 MPa was introduced into the tool from the fluid substance feed/discharge port 130 to hold the basal joint of the second finger in position, while the carrier gas injection port 150 and the gas output port 160 were held closed by means of rubber plugs. The number N of wrinkles of this example was 3 and the flexible member 120 used in this example satisfied the requirement of the formula (1). Then, the subject's hand from the wrist to the fingertips was held quietly in position in the vessel filled with 200 ppm isoprene gas for 10 minutes with the skin gas collection tool fitted to the hand. Thereafter, the hand from the wrist to the fingertips was drawn out from the vessel filled with isoprene gas, while the skin gas collection tool remained fitted, and a gastight syringe was driven into the gas output port 160 to draw out gas by 100 μL. The components of the drawn out gas were observed by means of a gas chromatograph-mass spectrometer. As a result, no isoprene gas was detected to confirm that the obtained skin gas was free from any external gas and hence the components analysis was not affected by external gas.

### Example-2

A casing 110, a fluid substance feed/discharge port 130, a skin gas collection vessel 140, a carrier gas injection port 150 and a gas output port 160 were prepared with SUS316. The inner diameter 2R of the casing was made equal to 78 mm. A tube having a thickness of 1.12 mm and an outer diameter of 78 mm and made of polyisoprene was used for a tubular flexible film member 120. The force σ required to expand a cube whose sides had a unit length and that was made of the material of the tubular flexible film member by a unit length was 0.042 MPa/mm. Thus, σt was 0.005 MPa.

A palm was inserted into a skin gas collection tool according to the present invention and compressed air of 0.008MPa was introduced into the tool from the fluid substance feed/discharge port 130 to hold the wrist in position, while the carrier gas injection port 150 and the gas output port 160 were held closed by means of rubber plugs. The wrist had a diameter 2r of about 45 mm. The number N of wrinkles of this example was 5 and the flexible member 120 used in this example satisfied the requirement of the formula (1). Then, the subject's arm from the elbow to the fingertips was held quietly in position in the vessel filled with 200 ppm isoprene gas for 10 minutes as in Example 1. Thereafter, the components in the skin gas collection tool were observed. As a result, no isoprene gas was detected to confirm that the obtained skin gas was free from any external gas and hence the components analysis was not affected by external gas.

Skin gas that is free from air can be collected with ease by means of a skin gas collection tool according to the present invention. Thus, the body conditions can be checked in a non-invasive and simple manner.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-225448, filed Oct. 13, 2011, which is hereby incorporated by reference herein in its entirety.

What is claimed is:

1. A skin gas collection tool comprising:
a casing forming a tubular opening section for insertion of a skin gas collection body part;
a tubular flexible film member arranged along the inner lateral surface of the casing; and
a gas collection vessel gastightly linked to the casing;
the tubular flexible film member being held at the opposite ends thereof in gastight contact with the inner lateral surface of the casing to form a sealed space between itself and the inner lateral surface of the casing;
the casing having a fluid substance feed/discharge port for feeding a fluid substance into or discharging a fluid substance from the sealed space;
the tubular flexible film member being forced to expand toward the center of the tube as a fluid substance is fed into the sealed space to thereby hold the tubular flexible film member in gastight contact with the skin gas collection body part inserted into the tubular opening section;
the product at of the thickness t of the tubular flexible film member and the force a required to stretch a cube of a unit length of the tubular flexible film member by a unit length in a direction satisfying the requirement of the inequality formula (1) shown below and expressed in terms of the limit pressure Pₘₐₓ of the fluid substance, the length s of the tubular flexible film member, the radius R of the tubular opening, the radius r of the region of the skin gas collection body part held in gastight contact with the tubular flexible film member as approximated by a circular cylinder and the number N of the folds produced on the flexible film member when the skin gas collection body part is held in position:
$$σ t \leq \frac{{P}_{max} s}{\sqrt{2} ( π - 2 ) {\{ \begin{matrix}{( R + r )}^{2} {sin}^{2} ( \frac{90 R - 90 r}{NR} ) + \\ {( R - r )}^{2} {cos}^{2} ( \frac{90 R - 90 r}{NR} )\end{matrix} \}}^{\frac{1}{2}}} . \qquad ( 1 )$$

2. The tool according to claim 1, wherein the skin gas collection tool is a tool for collecting gas emanated from the skin surface of a human being's hand or foot and the limit pressure Pₘₐₓ is 0.0087 MPa.
